(19) United States
(12) Patent Application Publication
(10) Pub. No.: US 2004/0009601 A1
(43) Pub. Date: Jan. 15, 2004

(54) Methods for the regeneration and transformation of cotton

(75) Inventors: Thea A. Wilkins, Woodland, CA (US); Huai-Yu Wang, London (CA); Rajiv Mishra, Davis, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: The Regents of the University of California, Oakland, CA (US)

(21) Appl. No.: 10/196,656

(22) Filed: Jul. 15, 2002

(51) Int. Cl. A01H 1/00; C12N 15/82; C12N 5/04

(52) U.S. Cl. 435/419; 800/278

(57) ABSTRACT

The present invention relates to improved methods of regeneration and Agrobacterium-mediated transformation of cotton via somatic embryogenesis.

### FIELD OF THE INVENTION

The present invention relates to improved methods of regeneration and Agrobacterium-mediated transformation of cotton via somatic embryogenesis.

### BACKGROUND OF THE INVENTION

Cotton (Gossypium spp.) is the world's leading natural fiber, a renewable resource, and the second largest oilseed crop. Cotton production is a multi-billion dollar industry, and therefore a vital agricultural commodity to both the U.S. and global economies. In addition to textile manufacturing, cotton and cotton by-products provide raw materials that are used to produce a wealth of consumer-based products, foodstuffs, livestock feed, fertilizer and paper. The production, marketing, consumption and trade of cotton-based products further stimulates the economy, and based on revenues in excess of $100 billion generated annually in the U.S. alone, cotton is the number one value-added crop. Approximately 90% of cotton's value resides in the fiber (lint), yet yield and fiber quality has declined, especially over the last decade (Meredith (2000), Proc. World Cotton Research Conference II, Athens, Greece pp.97-101). This downward trend has been attributed to general erosion in genetic diversity of cotton varieties, and an increased vulnerability of the crop to enviromental conditions (Bowman et al., Crop Sci. 36:577-581 (1996); Meredith, supra). In light of the critical need to increase diversity in the gene pool, cotton improvement programs are increasingly turning to the application of molecular approaches to breeding and germplasm utilization. With cotton biotechnology coming of age in 1996 with the large-scale commercial production of transgenic cotton in the U.S., genetic engineering figures to play a prominent role in improvement programs. The percentage of U.S. acreage devoted to genetically modified cotton continues to rise, and now accounts for more than 75% of the total cotton acreage. Transgenic cotton in commercial production is genetically modified for one or more input traits, that is, new traits that enhance agronomic performance for biotic and abiotic resistance. Genetic modification of cotton for output traits—traits that enhance food and fiber quality—is a prime target for future advances in cotton biotechnology. Besides the benefits to the consumer, genetic engineering for input and output traits will undoubtedly increase production efficiency, decrease production costs, lessen impact on the environment and improve sustainability (Willmitzer, Plant Cell Tiss. Organ Cult. 60:89-94 (1999)).

Cotton biotechnology hinges on two tightly interlaced processes—transformation and regeneration. Despite innovative technical advances, and the remarkable success stories in cotton biotechnology, the efficiency and genotype-dependence of regeneration are the two most limiting factors in the development of genetically modified cotton (Wilkins et al., Crit. Rev. Plant. Sci. 19:511-550 (2000)). Agrobacterium-mediated transformation and regeneration of cotton via somatic embryogenesis remains the preferred method of choice in this regard, as its advantages significantly outweigh the disadvantages relative to other methods (Wilkins et al., Crit. Rev. Plant. Sci. 19:511-550 (2000)). However, published methods require ˜10 to 12 months or longer to regenerate transgenic cotton plants (Firoozabady et al., Cell Dev. Bio. 29P: 166-173 (1987); Umbeck et al., Bio/Technology, 5:263-267 (1987); Lyon et al. Transgenic Research 2:162-169 (1993); Thomas et al., Plant Cell Reports 14:758-762 (1995); Trolinder and Goodin, Plant Cell Tissue Organ Cult. 12:31-42, 43-53 (1988), and as time in culture increases, so does somaclonal variation. Seed production to produce genetically stable transgenic lines requires an additional 6 to 8 months, meaning that each transgenic plant may take 2 years or more to develop. In addition, cotton regeneration is highly genotype-specific, and highly regenerable lines selected from the obsolete cultivar Coker 312 (Trolinder and Xhixian, Plant Cell Rep. 8: 133-136 (1989)) serves as the industry standard at this time, although linkage drag during introgression of transgenes into elite cultivars continues to be an issue of concern. Gene transfer or stacking of transgenes is accomplished primarily via backcrossing to elite cultivars, and given the significant proportion of cotton planted to transgenic varieties, the impact is to further dilute the gene pool and narrow genetic diversity in cultivated plants.

Because of the importance of the cotton industry in the United States and worldwide, a need exists for improved methods of cotton regeneration and transformation. In particular, a need exist for methods of increasing the range of genotypes that can be genetically modified thereby allowing for genotype-independent transformation and enhancement of genetic diversity in molecular breeding programs. The present invention meets this and other needs.

### BRIEF SUMMARY OF THE INVENTION

The present invention provides new methods for cotton regeneration and transformation.

In one aspect, the present invention provides a method for regenerating cotton. The method comprises the steps of providing a cotton explant selected from the group consisting of Gossypium, inducing callus formation in an induction medium comprising two or more auxins, selecting superior callus, and culturing the superior callus to form embryogenic callus. In one embodiment, the explants are selected from the group consisting of hypocotyl, cotyledon, immature zygotic embryos, leaves, anthers, petal, ovules, roots, meristems and mixtures thereof. In a second embodiment, the cotton is an Acala cotton variety. In a third embodiment, the Acala cotton variety is selected from the group consisting of Maxxa, Riata, and Ultima.

In another aspect, the auxins used in the methods of the present invention are selected from the group consisting of dichlorophenoxyacetic acid (“2,4-D”) and α-napthaleneacetic acid (“NAA”). In one embodiment, 2,4-D is present in the medium in concentrations between about 0.025 mg/L and about 0.1 mg/L. In a second embodiment, 2,4-D is present in the medium at about 0.05 mg/L. In a third embodiment, 2,4-D is present in the medium at about 0.1 mg/L. In a fourth embodiment, NAA is present in the medium in concentrations between about 1.5 mg/L and about 5 mg/L. In a fifth embodiment, NAA is present in the medium at about 1.5 mg/L. In a sixth embodiment, NAA is present in the medium at about 2 mg/L.

In another aspect of the present invention, the induction media of the present invention is free of cytokinins.

In another aspect of the present invention, the induction media is Murashige and Skoog medium and the carbohydrate source is glucose or sucrose. In one embodiment, the carbohydrate source is glucose and the glucose is at 30 g/L.

In another aspect of the present invention, the method for regenerating cotton further comprises transferring the embryogenic callus to a plant germination medium and culturing the embryogenic callus on the plant germination medium until a plantlet is formed.

In another aspect of the present invention, the method for regenerating cotton further comprises rooting the plantlet and developing fertile plants and seeds.

In another aspect of the present invention, the plant germination medium is Stewart's medium.

In another aspect of the present invention, calli are induced in light-dark cycles of about 16 hours of light and about 8 hours of darkness at a temperature from about 25 degrees Celsius to about 35 degrees Celsius. In one embodiment, the temperature is from about 26 degrees Celsius to about 30 degrees Celsius. In a second embodiment, the calli are induced in induction medium for about four to about six weeks.

In another aspect of the present invention, the step of culturing the superior callus to form embryogenic callus includes filtering and washing the cultures every two to three weeks.

In another aspect, the present invention provides a method for transforming cotton. The method for transforming cotton comprises the steps of providing a cotton explant selected from the group consisting of Gossypium, inducing callus formation in induction medium, suspending callus in suspension culture to break up the callus, injuring cells to produce single cells and small cell clusters, co-cultivating the cells with Agrobacterium wherein the Agrobacterium comprises a DNA sequence of interest and the DNA sequence of interest comprises a selectable marker, culturing cells under selection to select against Agrobacterium, and recovering transgenic cells. In one embodiment, the explants are selected from the group consisting of hypocotyl, cotyledon, immature zygotic embryos, leaves, anthers, petals, ovules, roots, meristems and mixtures thereof. In a second embodiment, the cotton is an Acala cotton variety. In a third embodiment, the Acala cotton variety is selected from the group consisting of Maxxa, Riata, and Ultima.

In another aspect, the induction media used in the methods for transforming cotton comprises two or more auxins. In one embodiment, the two auxins are selected from the group consisting of dichlorophenoxyacetic acid and a-napthaleneacetic acid. In a second embodiment, the medium is free of cytokinins. In a third embodiment, the medium is Murashige and Skoog medium and the carbohydrate source is glucose or sucrose.

In another aspect, the method for transforming cotton further comprises regenerating a cotton plant. In on embodiment, transgenic cells are cultured to produce somatic embryos.

In another aspect, the method for transforming cotton further comprises transferring the somatic embryos to plant germination medium and culturing the somatic embryos on the plant germination medium until a plantlet is formed.

In another aspect, the method for transforming cotton further comprises rooting the plantlet and developing fertile plants and seeds.

In another aspect, the present provides a cotton plant produced by a method comprising the following steps of providing a cotton explant derived from an elite cotton species selected from the group consisting of Gossypium hirsutum L., inducing callus formation in a medium comprising dichlorophenoxyacetic acid (“2,4-D”) and α-napthaleneacetic acid (“NAA”), selecting superior callus, and culturing the superior callus to form embryogenic callus.

In yet another aspect, the present invention provides a cotton plant produced by a method comprising providing a cotton explant selected from the group consisting of Gossypium, inducing callus formation in induction medium, suspending callus in suspension culture to break up the callus, injuring cells to produce single cells and small cell clusters, co-cultivating the cells with Agrobacterium wherein the Agrobacterium comprises a DNA sequence of interest and the DNA sequence of interest comprises a selectable marker, culturing cells under selection to select against Agrobacterium, and recovering transgenic cells.

### DETAILED DESCRIPTION OF THE INVENTION

A. General Overview

The present invention provides new methods of regenerating cotton. The present invention is based, in part, on the surprising discovery that the use of two or more auxins in a callus induction medium greatly increases the regeneration potential of cotton cultivars, in particular, elite cotton cultivars, e.g., Acala cotton.

The present invention also provides new methods of transforming cotton. The present inventors discovered for the first time that co-cultivating cotton cells with Agrobacterium, not at the explant stage, but after callus induction, greatly increases the efficiency of cotton transformation.

Accordingly, the present invention provides new and improved methods of creating genetically modified cotton cultivars.

B. Definitions

The phrase “nucleic acid sequence” refers to a single or double-stranded polymer of deoxyribonucleotide or ribonucleotide bases read from the 5′ to the 3′ end. It includes chromosomal DNA, self-replicating plasmids, infectious polymers of DNA or RNA and DNA or RNA that performs a primarily structural role.

The term “callus” refers to a disorganized mass of mainly undifferentiated cells produced as a consequence of plant tissue culture or wounding. “Superior calli” or “good quality calli” refer to calli with the potential to form shoots and roots and eventually regenerate into whole plants. Superior callus can be distinguished by a parrot-green/creamy color, soft and friable texture, readily dispersed cell clumps in liquid medium, and a nodular shape.

An “auxin” is any one of various usually acidic organic substances that promotes cell elongation in plant shoots and usually regulates other growth processes, e.g., indoleacetic acid. The term “auxin” also refers to a synthetic substance, e.g., 2,4-D, NAA, resembling indoleacetic acid in activity. Exemplary auxins include, but are not limited, to Naphthalene acetic acid (“NAA”), Indole-3-acetic acid (“IAA”), Indole-3-butyric acid (“IBA”), 2,4,-dichlorophenoxyacetic acid (“2,4-D”), Phenyl acetic acid (“PAA”), 4-chlorophenoxyacetic acid (“4-CPA”), 4-(2,4-dichlorophenoxy)butyric acid (“2,4-DB”), tris[2-(2,4-dichlorophenoxy)ethyl]phosphite (“2,4,-DEP”), (RS)-2-(2,4-dichlorophenoxy) propionic acid (“dichlorprop”), (RS)-2-(2,4,5-tiichlorophenoxy) propionic acid (“fenoprop”), 2-(1-napthyl)acetamide (“napthaleneacetamide”), (2-napthyloxy)acetic acid (“napthoxyacetic acid”), and (2,4,5-trichlorophenoxy)acetic acid (“2,4,5-T”).

The phrase “nucleic acid sequence encoding” refers to a nucleic acid which directs the expression of a specific protein or peptide. The nucleic acid sequences include both the DNA strand sequence that is transcribed into RNA and the RNA sequence that is translated into protein. The nucleic acid sequences include both the full length nucleic acid sequences as well as non-full length sequences derived from the full length sequences. It should be further understood that the sequence includes the degenerate codons of the native sequence or sequences which may be introduced to provide codon preference in a specific host cell.

The term “promoter” refers to a region or sequence determinants located upstream or downstream from the start of transcription and which are involved in recognition and binding of RNA polymerase and other proteins to initiate transcription. A “plant promoter” is a promoter capable of initiating transcription in plant cells. Such promoters need not be of plant origin, for example, promoters derived from plant viruses, such as the CaMV35S promoter, can be used in the present invention.

An “expression cassette” refers to a nucleic acid construct, which when introduced into a host cell, results in transcription and/or translation of a RNA or polypeptide, respectively. Antisense or sense constructs that are not or cannot be translated are expressly included by this definition.

C. Cotton Regeneration

The present invention provides methods for regenerating cotton. The methods include the steps of providing a cotton explant, inducing callus formation in induction medium, selecting callus, and culturing the callus. The majority of these steps are well known in the art.

Explant Preparation

A first step in cotton regeneration is explant preparation. A varied assortment of plant organs and tissues can be used in the methods of the present invention for the initiation of callus cultures. The particular tissue used is not critical to the invention. Exemplary tissues include hypocotyl, cotyledon, immature zygotic embryos, leaves, anthers, petals, ovules, roots, and meristems. In an exemplary embodiment of the present invention, the explant is taken from the hypocotyl or cotyledon.

Explants are excised from seedlings or adult plants using known methods, Plant Cell Biotechnology 1997, Klee et al., Ann. Rev. of Plant Phys. 38:467-486 (1987). For example, in one method, prior to germination, cotton seeds are sterilized, e.g., using a sharp sterile scalpel, and placed in germination medium. Hypocotyl explants can then be excised from seedlings under sterile conditions. In an exemplary embodiment of the present invention, explants are excised from seedlings 7-10 days old.

In an exemplary embodiment of the present invention, hypocotyl explants are excised from seedlings of Acala cotton. In other embodiments, explants are excised from seedlings of any Gossypium species, e.g., Gossypium hirsutum L, Gossypium barbadense, Gossypium herbaceum, and Gossypium arboreum.

Callus Induction

After explant excision, the explant is placed in a chemically defined callus induction medium comprising two or more auxins and grown under sterile conditions in order to induce callus formation. Standard plant regeneration methods, using modified induction media of the invention, are used in the methods of the present invention (Evans et al., Protoplasts Isolation and Culture, Handbook of Plant Cell Culture, pp. 124-176, MacMillilan Publishing Company, New York, 1983; and Binding, Regeneration of Plants, Plant Protoplasts, pp. 21-73,CRC Press, Boca Raton, 1985). In an exemplary embodiment, explants are plated and cultured in callus induction medium for 4-6 weeks, incubated at a temperature of about 25° C. to about 35° C. in light/dark cycles, and subcultured every 3-4 weeks.

As noted above, the present invention provides improved induction media. For the purposes of the present invention, the callus induction medium is any known callus induction medium supplemented with two or more auxins. In an exemplary embodiment of the present invention, the medium is a Murashige and Skoog medium containing either glucose or sucrose as a carbohydrate source. As demonstrated below, when induced on media comprising two or more auxins, explants from cotton cultivars not known to be regenerable, or known to be very difficult to regenerate, are capable of developing into embryogenic callus and plantlets. Any auxin can be used in the methods of the present invention, e.g., indole-3-acetic acid (“IAA”), 2,4-dichlorophenoxyacetic acid (“2,4-D) or napthylacetic acid (“NAA”). In some embodiments of the present invention, the induction medium will also comprise a cytokinin, e.g., kinetin.

In some embodiments, it may be essential to optimize either auxin type or auxin concentration for a particular genotype. In order to determine the optimal auxin type or concentration to be used in the induction medium, standard methods can be used. For example, a hormonal regime for induction of callus capable of undergoing somatic embryogenesis can be determined by using different types and concentration of auxins in induction medium and determining the proportion of explants that produce superior callus, e.g., callus capable of undergoing embryogenesis. Methods of determining good quality callus or superior callus are known in the art. For example, good quality callus can be determined by examining the color, texture, dispersiveness in liquid media, size, and shape of each callus. Typically, good quality callus has a parrot green/creamy color and soft and friable texture. Good quality callus also forms readily dispersed cell clumps in liquid medium and is nodular and grainy.

One method of determining optimal auxin concentration is by performing a factorial experiment using an Orthogonal Array L9 experiment design to determine the hormonal regime for superior callus induction, (Taguchi, Introduction to Quality Engineering, Tokyo, Japan (1986)). In an exemplary embodiment of the present invention, the induction medium comprises MS salts supplemented with myo-inositol, B5 vitamins, MgCl₂, and a carbon source, e.g., glucose or sucrose, at standard concentrations. By varying the amount of auxin, one at a time, in standard induction media, optimal concentrations of auxin for the production of superior callus can be defined. For example, a preferred induction medium comprises 2,4-D in amounts of about 0.025 mg/L to about 0.15 mg/L and NAA in amounts of about 1.5 mg/L to about 5 mg/L.

Culturing Callus to Undergo Somatic Embryogenesis

After explants are excised and cultured on the induction medium of the present invention to induce callus formation, calli are transferred to a second culturing medium. In an exemplary embodiment of the present invention, superior calli, e.g., friable, parrot-green/creamy calli, are transferred to the second culturing medium. Any standard embryogenesis culturing medium and/or methods can be used. The particular embryogenesis mechanism is not critical to the present invention. In an exemplary embodiment, MS medium is supplemented with myo-inositol, B5 vitamins, MgCl₂, glucose and KNO₃. The calli are cultured in the second culturing medium using known methods, e.g., at a temperature between about 25° C. to about 35° C. and in light/dark cycles (see U.S. Pat. Nos: 5,695,999, 4,672,035, Plant Cell Biotechnology 1997). In an exemplary embodiment of the present invention, superior calli are suspended in the second MS medium and develop into embryogenic suspension cultures within 3 to 6 weeks. One of skill in the art can determine the embryogenic potential of the calli by any of several well-known callus characteristics. For example, the accumulation of small amounts of anthocyanins in the cultures can be used as an indicator of embryogenic potential. Embryogenic cultures of good quality are a dirty, grayish-green color and contain somatic embryos at different stages of development when observed under a dissecting microscope.

The suspension cultures containing the calli are washed and filtered frequently, e.g., every 2-3 weeks, to promote embryogenesis and improve the quality of somatic embryos. Using the methods of the present invention, an embryogenic callus is selectively subcultured for continued differentiation, growth, and development of somatic embryos.

Plantlet Formation

Methods for the production of whole plants from embryos produced by embryogenic calli as described are well known. After embryogenesis, embryogenic cell clusters are routinely selected and germinated to promote root and shoot formation. Embryos may be selected depending upon their shape as somatic embryos in varying stage of development possess different shapes, e.g., globular, heart-shaped and torpedo. For example, heart-shaped embryos are cultured on dehydration medium before germination whereas torpedo stage and embryos with well-developed cotyledons are transferred directly to germination medium. Germinated embryos are then transferred to soil. Germinated embryos may be transferred into jars to established rooted plantlets prior to transfer to soil.

D. Cotton Transformation

The present invention also provides new methods of Agrobacterium-mediated transformation. Explants are prepared as described above.

In some embodiments of the present invention, after explant excision, the explant is placed in a chemically defined induction medium and grown under sterile conditions in order to induce callus formation. For cotton transformation methods, any known induction medium and any cotton variety, e.g., Coker, Acala, can be used. In an exemplary embodiment, the induction media of the present invention can be used.

After explants are excised and cultured on induction medium to induce callus formation, good quality callus, e.g., friable but not embryogenic, is broken up into single cell and small cell clusters. Any known method can be used to break up callus into single cells and small cell clusters. In some embodiments, cells can be mechanically injured. In one method, glass beads are added to a flask containing the friable callus. The flask containing the callus and the beads is placed on a magnetic stirrer, thereby breaking up the callus and mechanically injuring the cells according to standard technology.

Methods of co-cultivating cells with Agrobacterium are well known in the art. In the present invention, single cells and small cell cultures are co-cultivated with Agrobacterium after callus induction, but before regeneration, e.g., embryogenesis or organogenesis.

For transformation of plant cells using Agrobacterium, explants may be combined and incubated with the transformed Agrobacterium for sufficient time for transformation, the bacteria killed, and the transformed plant cells cultured in an appropriate selective medium for embryogenesis. Nucleic acid sequences of interest for transformation typically contain a nucleic acid sequence of interest fused to a regulatory sequence capable of transcription or transcription and translation in plant cells. Sequences for transcription and translation will generally encode a polypeptide of interest. Fused to the nucleic acid sequence of interest may be one or more markers, which allow for selection of transformed Agrobacterium and transformed plant cells. A number of markers have been developed for use with plant cells, such as resistance to chloramphenicol, kanamycin, the aminoglycoside invention, one or another marker being preferred depending on the particular host and the manner of construction.

After transformation, the plants are regenerated using established procedures, e.g., somatic embryogenesis or organogenesis. Using the methods of the present invention, transgenic plantlets can be recovered ready to transfer to soil in less than 10 months. In preferred embodiments, the plantlets are recovered in 5 months or less.

There are several possible ways to obtain the plant cells of this invention which contain multiple expression constructs. Any means for producing a plant comprising a construct having a nucleic acid sequence of the present invention, and at least one other construct having another DNA sequence encoding an enzyme are encompassed by the present invention. For example, the expression construct can be used to transform a plant at the same time as the second construct either by inclusion of both expression constructs in a single transformation vector or by using separate vectors, each of which express desired genes. The second construct can be introduced into a plant which has already been transformed with the first expression construct, or alternatively, transformed plants, one having the first construct and one having the second construct, can be crossed to bring the constructs together in the same plant.

The examples below are provided to illustrate the invention but not to limit its scope. Other variants of the invention will be readily apparent to one of ordinary skill in the art and are encompassed by the appended claims. All publications, patents, and patent applications cited herein are hereby incorporated by reference in their entirety.

### EXAMPLES

### Example 1

Seed Preparation

Commercial seeds of Acala cotton (Gossypium hirsutum L. cvs Maxxa, Riata and Ultima) and Coker 312 were surface sterilized in a solution of 20% bleach for 20 minutes, rinsed with sterile deionized water 4-5 times and dried for 30 minutes on sterile filter paper. Surface-sterilized seeds were placed on a modified Stewart's Germination Medium (SGM) containing Stewart's macro- and micronutrients and vitamins (Stewart and Hsu, 1977), 0.75 g 1⁻¹ MgCl₂, 5 g 1⁻¹ of sucrose, 2 g 1⁻¹ Phytagel (Sigma P-8169) and 5 g 1⁻¹ Difco Bacto-agar. The medium was adjusted to pH 6.8 prior to the addition of agar.

Individual seeds were germinated for 7-10 days on SGM (15 ml) in 25×150 mm culture tubes at 28±2° C. under continuous diffuse lighting from cool white fluorescent bulbs.

### Example 2

Callus Induction

Hypocotyl explants ˜5 mm in length were excised from 7-10 day-old seedlings under sterile conditions. Explants were placed in a 25×100-mm petri dish (one seedling per plate) containing ˜30 ml of callus induction medium, sealed with parafilm and cultured at 28±2° C. under a combination of cool white and full spectrum fluorescent lights with a 16-hour light/8-hour dark cycle for 3 to 4 weeks to induce callus formation.

Initial experiments were conducted using MS2NK (2 mg 1⁻¹ NAA substituted for 2,4-D in MS2DK, Trolinder and Goodin, 1987), a medium used for callus induction in the elite regenerable Coker 312 line. To optimize a callus induction medium for Maxxa, a factorial experiment using an Orthogonal Array L9 (3⁴) experimental design ((Taguchi, Introduction to Quality Engineering, Tokyo, Japan (1986)) was performed to determine the best hormonal regime for induction of callus capable of undergoing somatic embryogenesis. The optimal combination of NAA, 2,4-D and kinetin was evaluated at varying concentrations (Table 1) in basal MS medium (Murashige and Skoog, Physiol. Plant. 15:473-497 (1962)) in four independent experiments. In each experiment, 18 hypocotyl explants were excised from 24-48 individual seedlings to provide two explants per seedling per treatment. Each replicated treatment was evaluated for the ability to proliferate a friable callus that was yellowish-green to creamy in color, granular in texture, and readily dispersed in liquid medium. Calli characteristics were scored at 3 weeks and 6 weeks for color, texture, dispersiveness in liquid media, and cell/callus size as the parameters for evaluation. Based on these criteria, the optimized Maxxa callus induction medium (MCIM) selected for cotton regeneration is comprised of MS salts (GibcoBRL Cat no. 11117-074) supplemented with 100 mg 1⁻¹ myo-inositol, B5 vitamins (10 mg 1⁻¹ thiamine-HCl; 1 mg 1⁻¹ nicotinic acid; 1 mg 1⁻¹ pyridoxine), 0.75 g 1⁻¹ MgCl₂, 30 g 1⁻¹ glucose, 2.0 mg 1⁻¹ NAA and 0.05 mg 1⁻¹ 2,4-D. The medium was adjusted to pH 5.8 before adding agar (2.5 g 1⁻¹ Phytagel).

A factorial experiment using an orthogonal array L9 (3⁴) design was conducted to determine optimal conditions for callus induction and somatic embryogenesis by Maxxa hypocotyl explants. Different hormonal combinations of auxin (NAA and/or 2,4-D) and cytokinin (kinetin) were tested at varying concentrations (Table 1) in basal MS medium (Murashige and Skoog, supra). Each of the 9 replicated treatments was scored for callus initiation efficiency (Y) based on two components—(I) the mean of the number of explants producing good quality callus and (II) embryogenic potential, that is, the developmental transition of undifferentiated callus to somatic embryogenesis—expressed as a percent (Table 1). The production and proliferation of callus considered of “good quality” was based on four criteria: color, texture, dispersiveness in liquid media, and size/shape of undifferentiated cells. Within each category, each callus was scored with a rating of 1 to 4 based on the following parameters: Color—dark green (4) to parrot-green/creamy (1), Texture—hard and compact (4) to soft and friable (1), Dispersiveness—intact (4) to readily dispersed cell clumps (1) in liquid medium, Size/Shape—small cells/compact callus (<1 mm) (4) to nodular, “grainy” callus (>2 mm) (1). Good quality callus received ratings of “1” in each category. The embryogenic potential (II) of Y represents the ability of undifferentiated parenchymal cells to organize into meristematic centers and successfully undergo somatic embryogenesis. The factors considered in determining II/Y included the formation of organized cell clusters in embryogenic cultures, the types of embryos produced (globular, heart-shaped, torpedo), and the color of the somatic embryo (white/transparent vs. yellow/opaque). The regeneration potential (RG), expressed as a percent, was calculated from mean Y values for each of the various hormonal combinations tested (factors (1), (2) and (3) in Table 1). The 3RG (Table 1) determined for each of the hormone treatments indicated that 2,4-D is essential for callus induction from Maxxa hypocotyls, while NAA is highly beneficial. In the absence of hormones, or in the presence of NAA alone, a friable callus was also initiated, albeit very slowly and requiring a much longer time in culture than other treatments which induced callus formation within 30 to 60 days. Based on the 3RG for kinetin, incorporation of cytokinin in the callus initiation medium was not critical for the induction of embryogenic callus from Maxxa hypocotyls.

The results of the factorial experiment indicated that the combination of two auxins (2,4-D and NAA) at concentrations of 0.05 mg 1⁻¹ and 2.0 mg 1⁻¹, respectively, was superior (Table 1,factor treatment 6) for inducing good quality callus and embryogenic cultures compared to all other treatments evaluated. The modified MS medium supplemented with 2,4-D and NAA was given the designation MCIM (Maxxa Callus Initiation Media). A few plants were successfully regenerated from embryogenic cultures of treatment 6 only, confirming that formation of good quality callus is key to successful regeneration. MCIM was used in subsequent regeneration experiments.

TABLE 1
Optimization of a modified callus initiation medium (MCIM) for
Acala cotton Gossypium hirsutum L. cv. Maxxa using an orthogonal
array L9 (3⁴) experimental design (Taguchi, supra)

| | A | | B | | C | | Callus | | |
|---|---|---|---|---|---|---|---|---|---|
| Treat- | 2,4-D | | NAA | | Kinetin | | Initiation Efficiency* | | |
| ment | mg I⁻¹ | | mg I⁻¹ | | mg I⁻¹ | | I (%) | II (%) | Y (%) |
| 1 | (1) | 0 | (1) | 0 | (1) | 0 | 0 | 0 | 0 |
| 2 | (1) | 0 | (2) | 1.0 | (2) | 0.5 | 16.7 | 0.4 | 8.55 |
| 3 | (1) | 0 | (3) | 2.0 | (3) | 1.0 | 0.8 | 16.7 | 8.75 |
| 4 | (2) | 0.05 | (1) | 0 | (2) | 0.5 | 0 | 0.4 | 0.2 |
| 5 | (2) | 0.05 | (2) | 1.0 | (3) | 1.0 | 95.8 | 25.0 | 85.4 |
| 6 | (2) | 0.05 | (3) | 2.0 | (1) | 0 | 100 | 100 | 100 |
| 7 | (3) | 0.1 | (1) | 0 | (3) | 1.0 | 45.8 | 45.8 | 45.8 |
| 8 | (3) | 0.1 | (2) | 1.0 | (1) | 0 | 95.8 | 95.8 | 95.8 |
| 9 | (3) | 0.1 | (3) | 2.0 | (2) | 0.5 | 91.7 | 87.5 | 89.6 |
| Σ RG1 | | 5.77 | | 15.33 | | 63.27 | | | |
| Σ RG2 | | 61.87 | | 63.25 | | 32.78 | | | |
| Σ RG3 | | 77.07 | | 66.12 | | 46.65 | | | |
| Σ RG | | 71.30 | | 50.79 | | 32.49 | | | |

Parenthesis in columns A, B and C represent the three concentrations of a particular hormone used in the factorial experiment (factors 1, 2 and 3)
RG - regeneration potential calculated from Y(%) using factor (1, 2 or 3) designation in corresponding column (A, B or C): Σ RG1 = mean of factor 1; Σ RG2 = mean of factor 2; Σ RG3 = mean of factor 3; Σ RG = mean of Σ RG1, Σ RG2 and Σ RG3 by column A, B or C
*I - percent of explants producing highest scoring callus based on color, texture, dispersiveness, and size;
II - embryogenic potential, percent of calli producing somatic embryos;
Y - mean of I and II

### Example 3

Regeneration of Acala Cotton

Hypocotyl explants excised from individual seedlings were cultured on MCIM to induce callus formation as described previously, a process requiring 4 to 6 weeks. Friable, parrot-green/creamy calli were selected and transferred to liquid MSK medium (MS salts supplemented with 100 mg 1⁻¹ myo-inositol, B5 vitamins, 0.75 g 1⁻¹ MgCl₂, 30 g 1⁻¹ glucose and 1.9 g 1⁻¹ KNO₃) adjusted to pH 5.8 (Trolinder and Goodin, Plant Cell Rep. 6:231-234 (1987)) using 0.5 g callus per 15-ml of medium. Liquid cultures in 125-ml flasks were incubated at 120 rpm under diffused light from cool white and full spectrum fluorescent lights at 28±2° C. as described previously, and subcultured every 3 to 4 weeks.

Cytoplasmically-dense embryogenic suspension cultures developed in 4-6 weeks. Three fractions were obtained after sieving suspension cultures through a series of sterile 20/30-, 50- and 100-mm mesh screens. Embryogenic suspension cultures considered to be of good quality were characterized by a dirty, grayish-green color and containing somatic embryos at different stages of development when observed under a dissecting microscope. The term “residue” refers to the cells retained by the screen, while the flow-through is called the “filtrate”. Large cell clumps and cell debris removed from the 20/30-mesh residue were discarded. The 20/30-mm mesh residue was washed twice with MSK medium, then suspended in MSK at a cell density of 40 mg ml⁻¹ and pipetted onto semi-solid MSK medium (MSK containing 2.5 g 1⁻¹ Phytagel) in 25×100-mm petri plates (2 ml plate⁻¹) to promote further development of somatic embryos. The 20/30-mesh filtrate was sieved through a 50-mm mesh screen, and embryogenic cell clusters and somatic embryos retained as the 50-mesh residue was likewise washed, suspended and plated on semi-solid MSK medium. The 100-mm mesh residue containing small cell clusters and individual cells collected from the 50-mm mesh filtrate was used to establish embryogenic maintenance suspension cultures. The 100-mm mesh residue was suspended in liquid MSK medium in 125-ml flasks and cultured for 4-6 weeks before repeating the sieving/plating steps. The 100-mm mesh filtrate was discarded. The embryogenic suspension cultures could be maintained by subculturing every 2 to 4 weeks.

After plating on semi-solid MSK medium, residual moisture was absorbed for 24 hours in a laminar flow hood. The plates were then sealed with parafilm and moved to a temperature-controlled culture room at 28±2° C. and 16 h light/8 h dark cycle using cool white and full spectrum fluorescent lights. After the first plating on semi-solid MSK medium, somatic embryos formed in suspension culture began to grow and develop. The callus became increasingly embryogenic, producing more somatic embryos over a 4 to 6 week interval. Development of somatic embryos is asynchronous and embryos at all stages could be easily observed at this time.

The 20/30- and 50-mesh residues plated on semi-solid MSK medium were maintained by subculturing every 4 to 6 weeks to promote the continuing formation of somatic embryos. The material selected for periodic subculturing included tiny, round, friable masses of embryogenic callus that were yellowish-green to creamy in color. Embryogenic calli were dispersed in liquid MSK medium and plated by pipetting 2 ml of cell suspension per petri plate of semi-solid MSK medium (“second” plating). Alternating cycles between semi-solid and liquid media allowed long-term maintenance of the embryogenic potential of the cultures although somaclonal variation and recovery of sterile plants becomes problematic as the length of stay in culture increases.

Somatic embryos at varying stages of development (globular, heart-shaped and torpedo) develop in the suspension cultures and on semi-solid media after 1-2 subculturing steps. Heart-shaped somatic embryos (≧5 mm) were dehydrated on Stewart's Dehydration (SD) medium (SGM supplemented with 10 g 1⁻¹ Bacto-agar) for 10-15 days in unsealed petri plates in the dark at 28±2°C. Although heart-shaped embryos were preferred for transfer to SD at this stage, globular embryos were also selected. Dehydrated somatic embryos were placed on SGA (SGM supplemented with 1.0 mg 1⁻¹ IAA) medium to promote rooting under cool white and full spectrum fluorescent lights at 28±2° C. Older, brown roots that developed on SGA medium were removed to enhance shoot development. Germinating embryos were transferred to fresh SGA medium every 6-8 weeks. When at least two true leaves developed, the plantlets were transferred to SGA medium (45 ml) in sterile pint jars. Once 4-6 leaves developed and a sufficient root system established, on average, in 4 to 6 weeks, plantlets were potted in soil (Pro-Mix, Premier Horticulture, Inc.), covered with a polyethylene bag and transferred to a growth chamber (30-35° C., 60% relative humidity). Plants were hardened off by gradual exposure to the environment by making incisions in the protective plastic bag. After 10-15 days, regenerated plants were transferred to the greenhouse for seed production from self-pollinated flowers. The Maxxa regeneration procedure was repeated using commercial seed of California Acala cotton cultivars Riata and Ultima.

### Example 4

Selection of Maxxa Elite Highly Regenerable Lines

Shoot tips excised from the same Maxxa seedlings used for hypocotyl explants (see above) were rooted on SGA in pint jars to produce meristem-derived plantlets within 4-6 weeks. Each meristem/shoot tip was assigned an identification number that corresponded to the cell culture line also derived from the same seedling. Plantlets were transferred to the greenhouse as described previously for seed increase of lines selected for regeneration potential, defined by the ability to produce 1) friable callus and 2) embryogenic cultures, and to 3) regenerate plants. Seeds (R2) were collected only from shoot meristem-derived R1 plants of lines also producing somatic embryos and regenerated plants. The R2 seed from 7 regenerable Maxxa lines (designated as Max-R1, -R2,and -R4 through -R8) were subjected to a second round of screening for regeneration potential by repeating the regeneration process on 4 to 6 R2 seeds for each R line, including the recovery and rooting of shoot tip/meristems. R3 seeds were harvested from Max-R meristem-derived plants grown to maturity in the greenhouse.

### Example 5

Results

Given the increasing percentage of transgenic cotton being grown worldwide, it is imperative that genotype-independent methods for regenerating cotton be developed to maintain diversity in the gene pool. Culture of hypocotyl explants (cv. Coker 312) on MS2NK callus induction medium, which serves as the industry standard for cotton regeneration and transformation, produces a characteristic yellowish-green, friable callus that is capable of undergoing somatic embryogenesis (reviewed in Wilkins et al., supra). In contrast, elite Acala-type cotton cultivars, prized for their high fiber quality and yield, have proven especially recalcitrant to regeneration. Compared to Coker callus, G. hirsutum L. cv. Maxxa hypocotyl explants cultured on MS2NK medium produce a hard, dark green, non-friable callus. This type of non-friable callus does not differentiate into embryogenic cultures, and thus plants cannot be regenerated. (Trolinder and Xhixian, Plant Cell Rep. 8:133-136 1989; Firoozabady and DeBoer, In Vitro Cell. Dev. Biol. 29P:166-173; Koonce et al., Beltwide Cotton. Prod. Res. Conf 2:1173 (1996); Sakhanokho et al., Beltside Cotton Prod. Res. Conf. 1:590-593 (1998), Beltside Cotton Prod. Res. Conf 1:570-575 (2000)); Nobre et al., Plant Cell Rep. 20:8-15 (2001)).

Individual Maxxa seeds were screened for the ability to regenerate fertile plants via somatic embryogenesis using MCIM to initiate callus. The major steps in regenerating Maxxa plants are depicted in Table 2. Hypocotyl explants excised from Maxxa seedlings and cultured on MCIM initiated callus in 3 to 4 weeks (Table 2). The selection of good quality callus, as defined by the factorial experiment, was critical to the successful establishment of embryogenic cell suspension cultures in hormone-free media within 3 to 4 weeks (Table 2). The accumulation of small amounts of anthocyanins (red pigmentation) in callus and embryogenic cultures was a good indicator of embryogenic potential. Removal of cell debris by filtering and washing the cell suspension cultures every 2-3 weeks promoted embryogenesis and considerably improved the quality of somatic embryos. Embryogenic suspension cultures considered to be of good quality were characterized by a dirty, grayish-green color, and frequently contained somatic embryos at different stages of development when observed under a dissecting microscope. Suspension cultures were sieved and embryogenic cell clusters and small globular and heart-shaped embryos retained as the 20/30- and 50-mesh residues were plated on solid medium at densities that promoted somatic embryogenesis in pro-embryonic cultures and somatic embryo development. Plating cell density was an important consideration as high plating densities resulted in non-embryogenic callus formation, and in some cases, de-differentiation of pro-embryogenic cultures into callus. Embryogenic cell clusters sieved from the 50-mm filtrate and retained as the 100-mm reside served as starting material for embryogenic maintenance suspension cultures. After 4 to 6 weeks, the maintenance suspension cultures were sieved and plated on semi-solid MSK for development of somatic embryos. Subculturing every 2 to 4 weeks maintains the embryogenic suspension cultures. On semi-solid MSK medium, opaque, cytoplasmically-dense somatic embryos were present at various stages of development, including globular, heart- and torpedo-shaped embryos that developed approximately 6 to 8 weeks following plating of sieved embryogenic suspension cultures. Due to the asynchronous nature of somatic embryogenesis in these cultures, globular embryos and embryogenic cell clusters were selected and subcultured every 4 to 6 weeks for continued differentiation, growth and development of somatic embryos (Table 2).

Heart-shaped embryos were routinely selected, cultured on dehydration medium to mimic seed dormancy by decreasing the moisture content, and “germinated” on SGA medium to promote root and shoot formation (Table 2). Older somatic embryos (e.g., torpedo stage and embryos with well-developed cotyledons) by-passed the dehydration step by being transferred directly to germination medium. Germinated embryos were transferred into pint jars to establish rooted plantlets prior to transfer to soil and the greenhouse (Table 2). The morphology, growth and development, pollen fertility and seed set of regenerated plants was indistinguishable from Maxxa control plants.

TABLE 2
Stages in the regeneration of cotton (Gossypium hirsutum L.).

| Stage | Activity | Medium | Time |
|---|---|---|---|
| I | Seedling germination | SGM | 7-10 days |
| II | Preparation of ˜5-mm hypocotyls | — | 2 hours |
| | explants | | |
| III | Callus induction* | MCIM | 3-4 weeks |
| IV | Embryogenic suspension cultures | MSK | 6-8 weeks |
| | Optional: subculture | MSK | 2-4 weeks |
| V | Sieving suspension cultures | MSK | 2 hours |
| VI | Plating of washed 30/50-mesh cultured | Semi-solid | 2 hours |
| | cells | MSK | |
| VII | Somatic embryogenesis | Semi-solid | 4-8 weeks |
| | | MSK | |
| VIII | Dehydration of heart-shaped and | SD | 7-15 days |
| | torpedo somatic embryos (optional) | | |
| IX | Germination of somatic embryos | SGA | 6-8 weeks |
| X | Growth of plantlets in pint jars | SGA | 4-6 weeks |
| XI | Plant transferred to soil | — | 10-15 days |

*The ‘Coker’ callus induction medium is MS2NK (N. Trolinder, unpublished data)

Based on the eight fertile regenerated (R1) lines producing seed from hypocotyl explants of individual seedlings, the regeneration potential in Maxxa commercial seed was estimated at 17.4% (Table 3). The successful transition from callus to somatic embryogenesis and ultimately, the successful regeneration of plants was dependent on medium composition, hormone regimes and culture conditions that would initiate callus capable of undergoing somatic embryogenesis. As indicated earlier, all attempts to utilize the MS2NK “Coker” callus induction medium met with failure. However, the very opposite was true for Coker hypocotyl explants cultured on MCIM. MCIM-cultured Coker explants initiated and proliferated good quality callus that differentiated into highly embryogenic cell lines (data not shown). These results suggest that MCIM expands the number of genotypes capable of regeneration.

The regeneration potential (RG) of regenerated Maxxa (R1) lines was determined in the R2 generation by subjecting individual seedlings derived from six meristem-derived R1 plants to a second cycle of selection. R2 seeds for each of six regenerated Maxxa lines (R2,R10, R15, R23, R34, R43; Table 3) were germinated to provide meristem/shoot-tips and hypocotyl explants to repeat the selection and regeneration process. Each of the R2 seedlings (100%) tested for the six R lines produced good quality callus from hypocotyl explants cultured on MCIM, and in almost all cases, successfully differentiated into embryogenic cultures (Table 3). Once established, somatic embryos were selected and germinated to produce plantlets from each cell line.

After one cycle of selection, every seedling tested in three of the R lines (R10, R34, R43) successfully underwent somatic embryogenesis, from which fertile plants were recovered. In these lines, RG is 100% and these plants are considered homozygous, and RG is thereby genetically stable and maintained. These results showed that there was positive selection for RG. However, the number of seedlings successfully producing somatic embryos and fertile regenerated plants varied among the lines (Table 3). In three other R lines (R2, R15, R23), RG ranged from a low (50%) in R15 to a high of 83.3% in R2 (Table 3). However, RG still increased significantly in each line compared to that of the original population of commercial seeds (17.4%), indicating positive selection for RG among these lines as well. The fact that these lines did not attain 100% RG after one cycle of selection as in the other R2 lines is the likely result of genetic factors, although culture conditions may be contributing factors and cannot be excluded from consideration as RG is a multigenic trait and subject to environmental variation (Gawel and Robacker, Euphytica, 49:249-254 (1990); Kumar et al., Plant Cell Rep. 18:59-63 (1998)). Nevertheless, RG in these lines is expected to approach 100% in the R3 generation following the second cycle of selection performed in this study.

When viewed from the population as a whole, RG significantly increased from an average of 17.39% in commercial Maxxa seed to a mean of 84% in R1 lines (Table 3), an increase in RG of over 65% after a single cycle of selection. The regenerated Maxxa lines were assigned germplasm designations Max-R1 through Max-R6 (Table 4). R3 seeds harvested from meristem-derived R2 Max-R lines will be used for seed increase.

TABLE 3
Regeneration potential (RG) of elite Acala cotton (Gossypium
hirsutum L.) cultivars

| | | | | No. | Regeneration |
|---|---|---|---|---|---|
| | No. | No. | No. | Cell Lines | Potential |
| Cultivar/ | Seed- | Producing | Embryogenic | Producing | (RG) |
| Lines | lings | Callus* | Lines | Plants | % |
| Maxxa | 46 | 46 | 8 | 8 | 17.41¹ |
| Maxxa | 32 | 32 | 27 | 27 | 84.4² |
| R2 | 6 | 6 | 5 | 5 | 83.3 |
| R10 | 6 | 6 | 6 | 6 | 100 |
| R15 | 4 | 4 | 2 | 2 | 50 |
| R23 | 6 | 6 | 4 | 4 | 66.7 |
| R34 | 4 | 4 | 4 | 4 | 100 |
| R43 | 6 | 6 | 6 | 6 | 100 |
| Riata | 15 | 15 | 12 | 12 | 80.0 |
| Ultima | 18 | 18 | 8 | 8 | 44.4 |

¹Mean RG following first cycle of selection in commercial seed
²Mean RG following second cycle of selection in six regenerated R1 lines (R2, R10, R15, R23, R34, R43)

TABLE 4
Independent Regenerated Lines of
Gossypium hirsutum L. cv. Maxxa

| Regenerated | Germplasm |
|---|---|
| Cell Lines* | Designation |
| R2 | Max-R1 |
| R10 | Max-R2 |
| R15 | Max-R3 |
| R23 | Max-R4 |
| R34 | Max-R5 |
| R43 | Max-R6 |

*R2 lines recovered from second cycle of selection among R1 plants for regeneration potential (RG)

To test MCIM on other Acala genotypes, and estimate regeneration potential (RG), commercial seed of the cultivars Riata and Ultima were put into culture and regenerated. Each step in the process was evaluated using the same criteria established in the MCIM factorial experiment (Table 1). For Riata, hypocotyl explants produced high quality callus well within an acceptable time frame (Table 2). A fast-growing non-embryogenic callus became embryogenic after 2 to 3 subcultures. Yellowish-green in color, Riata callus was grainy and bore anthocyanin pigmentation and was readily dispersed in suspension cultures while still in the callus induction phase. Proliferating embryogenic cultures produced high-quality somatic embryos that germinated and produced plantlets. RG for Riata, calculated from the number of seedlings producing regenerated plants, was 80%, a value that was considerably higher Maxxa RG (17.4%) in commercial seed, and was as high, or higher than some Max-R lines after one cycle of selection (Table 3). However, Riata RG was not overly surprising given its Coker 312/Maxxa pedigree.

The Acala cultivar Ultima has a different genetic background than Maxxa, and when put through the regeneration process, somatic embryogenesis was essentially stalled for the most part at the callus induction stage. This problem was eventually overcome by supplementing MCIM with kinetin (0.1 mg 1⁻¹). Once good quality callus was formed, the subsequent stages proceeded as expected, resulting in embryogenic cultures and formation of somatic embryos, which were subsequently germinated into plantlets. Ultima RG (44.4%) was ˜2-fold higher than Maxxa RG, but only one-half that of Riata RG (Table 3). These results reinforce RG genetic variation and heterogeneity of RG in the cotton gene pool.

Stage IV in the regeneration schema entailed establishing a suspension culture from callus tissue, a process that amplifies embryogenic cells and facilitates differentiation and development of quality somatic embryos. However, the success rate for germination of somatic embryos into plantlets that survive to the greenhouse is very low compared to the hundreds of somatic embryos produced for each cell line. It was found that decreasing the concentration of sucrose in SGA rooting medium improved the number of somatic embryos successfully regenerated to ˜6-8% (data not shown), a number that is equitable to the 5-6% efficiency reported for recovery of Coker plantlets (reviewed in Wilkins et al., 2000, supra).

Discussion

The cultivation of transgenic cotton (G. hirsutum L.) rapidly gained significant ground in the late 1990's, and now accounts for the majority of cotton in production in the U.S. and many other countries. Yet, despite the commercial success of genetically modified cotton, the transformation and regeneration of cotton via somatic embryogenesis is not a trivial process by any means, and cotton remains one of the more recalcitrant species to manipulate in culture (reviewed in Wilkins et al. 2000, supra). Embryogenic potential is a polygenic, low heritable trait (Gawel and Robacker, supra; Kumar et al., supra) that is highly-genotype dependent as reported here and elsewhere (Trolinder and Xhixian, 1989 supra; Firoozabady and DeBoer, 1993 supra; Koonce et al., 1996 supra; Sakhanokho et al., 1998 supra, 2000; Nobre et al., 2001 supra). Selection for embryogenic potential in Coker 312, an obsolete cultivar adapted for cotton production in the southeastern region of the U.S., produced a highly regenerable line for introducing transgenes. The most efficient method for producing high-performing transgenic cultivars at present relies on first introducing transgenes into Coker 312, and the subsequent transfer of the transgene into elite genotypes via a backcross program. By keeping the number of backcross generations to a minimum (≦3), the result is a net gain of a Coker genetic background in the gene pool of cultivated cotton varieties. Linkage drag is therefore certainly an issue as deleterious alleles, or those unfavorable to agronomic traits, are introduced into the gene pool. Efforts to surmount the genotype-dependent barrier using Agrobacterium-mediated transformation or particle gun bombardment of meristems have met with limited success.

This study was undertaken to develop regenerable cotton germplasm from elite Acala cotton cultivars—high-yielding varieties prized for their high fiber quality. The underlying premise being that genotype-independent transformation and regeneration allows for the introduction of transgenes directly into elite genetic backgrounds, and satisfies increasing demands for the rapid delivery of improved cultivars into commercial production. This study was highly successful in this regard, in that several regenerable lines of elite Acala cotton cultivars have been developed to facilitate forward breeding and eliminate, or at the very least, limit the need for backcross breeding programs. At the same time, these regenerable Acala lines promise to increase efficiency of developing transgenic cotton plants while simultaneously decreasing production costs, and bear significant ramifications for improvement strategies in molecular breeding programs.

The transformation and regeneration of cotton are two interdependent processes. Cotton regeneration is heavily influenced by a number of factors, and is especially sensitive to environmental variation as regeneration potential (RG) is a multigenic trait (Gawel and Robacker, 1990 supra; Kumar et al., 1998 supra). The regeneration potential (RG) for any given genotype, as defined here, requires formation of a friable callus from explants that undergo successful morphogenesis to produce somatic embryos, and the eventual recovery of intact, fertile plants. The first step to successful regeneration is callus formation. The Coker callus induction medium supports formation of friable, embryogenic callus on a limited number of genotypes, including a few Acala cottons (Wilkins et al., 2000 supra and references therein), while many other genotypes, including those used in this study, fail to initiate callus or produce hard, non-friable calli. We therefore set out to define cultural parameters that would induce friable callus from genotypes for which the Coker induction medium proved unsuitable. Using the elite Acala cotton cultivar ‘Maxxa’ as the test genotype, and Coker 312 as a control, a number of media were evaluated and discarded in preference for a MS basal medium. A factorial experiment designed to determine optimal hormonal regimens revealed that the unique combination of two different auxins (NAA and 2,4-D) supported the initiation and proliferation of high quality callus from both Maxxa and Coker hypocotyls explants using color, size and texture, organization (friability), and lastly, regeneration potential as evaluation criteria. The empirically determined media formulation suitable for Maxxa was designated as Maxxa callus induction medium (MCIM) to distinguish it from other cotton media. Cytokinin was not a requirement for either Maxxa or Coker callus formation in the presence of not just one, but two synthetic auxins. The regeneration potential of Maxxa and other Acala cottons, as determined by the differential growth of callus on Coker medium and MCIM, is believed to reflect allelic differences influenced by the genetic background. This contention is supported by another elite Acala cotton cultivar's (Ultima) need for supplemental kinetin in MCIM in order to efficiently produce embryogenic callus.

Callus characteristics—color, texture, friability, and size—all play a major role in the successful regeneration of cotton via somatic embryogenesis. The best callus, initiated on MCIM and later becoming embryogenic, was light parrot-green in color and featured a grainy, nodular texture. In contrast, the preferred callus for selection using the Coker procedure is usually light cream in color. In callus initiated on either the Maxxa or Coker callus induction media, small amounts of red pigmentation resulting from the accumulation of anthocyanins is a good indicator of high quality callus. Grainy-textured callus capable of undergoing somatic embryogenesis tends to consist of larger, proliferating callus ≧2 mm in size and made up of large cells loosely organized in a friable callus. Hard, tightly organized, compact calli are non-friable and non-embryogenic. Sunilkumar and Rathore (Molec. Breeding 8:37-52) reported a similar requirement for larger-sized callus in the recovery of transgenic plants. Another key factor in the regeneration process is plating density, especially during callus induction/proliferation and somatic embryogenesis, and in establishing embryogenic suspension cultures. If plating density is too low, further growth and development is aborted, whereas at a high plating density, embryogenic cultures de-differentiate into callus.

Once high quality callus is obtained, the transition to somatic embryogenesis and recovery of regenerated plants is reasonably straightforward (Troliner and Goodin, Plant Cell Rep 6:231-234, Plant Cell Tiss. Organ Cult. 12:31-42, Plant Cell Rep. 8133-136). The incorporation of improvements and refinements reported here resulted in the recovery of regenerated plants in as little as six months (Table 2), or in ˜40-50% of the time required in most published procedures (reviewed in Wilkins et al., 2000 supra). This method does, however, take ˜8 months, on average, to recover the desired number of independent cell lines, mainly because asynchronous development of somatic embryos takes place over a 3 to 4 month period—a process supported by continued subculturing of embryogenic lines. A recently published protocol reports a similar timeline in the transformation and regeneration of Coker 312 (Sunilkumar and Rathore, 2001, supra). The condensed timeframe in this and other cotton regeneration procedures has virtually eliminated the problems associated with somaclonal variation that plagued the early generations of regenerated cotton (Stelly et al., Genome 32:762-770 (1989)).

In this study, considerable progress has been made towards genotype-independent regeneration of cotton via somatic embryogenesis with applications in Agrobacterium-mediated transformation. Regeneration of elite Acala cotton cultivars was successful using the method described here, although these very same lines exhibited no embryogenic potential whatsoever with the Coker method. In contrast, however, embryogenic potential of Coker 312 is very high using the Maxxa protocol in this study. A few varieties show embryogenic potential using the Coker procedure, although not to the level observed by Coker 312 and its sister lines. The method described here, initially developed, tested and evaluated for Maxxa, successfully harnessed the embryogenic potential in several Acala cotton cultivars as well as Coker 312 lines, meaning that the number of genotypes that can be effectively transformed and regenerated has been expanded to include elite, top-performing germplasm.

The regeneration potential among commercial seed lots for the Acala cotton cultivars Maxxa, Riata, and Ultima, was 17.4%, 80%, and (44.4)%, respectively. The Riata RG is about three times higher than that of Maxxa, but is easily explained by Riata's pedigree. Riata is a transgenic Round-up Ready® cotton developed by introgressing the herbicide resistance gene via backcrossing to Maxxa as the recurrent parent. Thus, Riata not only shares a similar genetic background, but has essentially been selected for RG, albeit indirectly, via crossing to a regenerable (transgenic) line and selecting for the input trait. Ultima has a different pedigree, and developed embryogenic callus on MCIM very poorly unless the medium was supplemented with a cytokinin. The different cultural requirements had a direct bearing on the RG estimated for Ultima, and underscores the genetic and environmental components of RG phenotypic variation.

Genotype-independent transformation and regeneration of elite cultivars accelerates the commercial release of genetically modified cotton in two ways. One, the time required to transfer the transgenic trait into adapted advanced breeding lines in backcross programs is minimized. The second advantage is that linkage drag, or the transfer of deleterious alleles from non-commercially important transgenic genotypes in backcrossing to elite recurrent parents is avoided. The results reported here concur with other studies (Trolinder and Xhixian, 1989 supra; Firoozabady and DeBoer, 1993 supra; Koonce et al., 1996 supra), clearly indicating a diverse range in phenotypic and genotypic variability for regeneration potential. In this study, identification and selection for RG genotypes yielded highly regenerable cotton (Max-R) lines in an elite genetic background (Mishra et al., Plant Cell Tissue and Organ Culture (unpublished)) in as little as one generation, despite reports of low RG heritability (Gawel and Robacker, 1990 supra; Kumar et al., 1998 supra). Of the Max-R lines carried forward, RG is 100% in one-half of the Max-R lines, meaning that the line is homogeneous and every seedling is capable of being efficiently transformed and regenerated. Transgenic Max-R somatic embryos have been produced, providing preliminary evidence for successful transformation of this elite germplasm (Mishra and Wilkins, unpublished data). A few Max-R lines required two to three cycles of selection to fix RG alleles, providing further confirmation for genotypic differences among the RG lines initially selected. The higher than expected RG found in the genetically modified Acala cultivar Riata is compelling evidence in support of this contention. Thus, selection for RG can be accomplished in a few generations once culture conditions are defined.

Genotype-specific regeneration potential includes genic control of two components—embryogenic and regeneration potentials. Despite the low heritability of embryogenic potential, there is sufficient genotypic variability within a given cultivar to warrant screening for RG among individuals for the purpose of developing elite regenerable lines for top-producing varieties. This study successfully increased the range of cotton genotypes that can be efficiently transformed and regenerated, developed highly regenerable lines from elite cultivars, and decreased the regeneration time to as little as six months. The highly regenerable Max-R germplasm developed in this study has immediate use and applications to the biotechnology industry. Selection for RG in advanced breeding lines and/or including elite regenerable lines as parents will increase the number of RG alleles in the gene pool, thereby moving the industry that much closer towards genotype-independence transformation and decreasing costs of generating genetically-modified cotton.

As molecular breeding programs become increasingly reliant on marker-assisted breeding, the challenge will be to identify DNA molecular markers for RG, a multigenic trait with low genetic and high environmental variability. Three strategies are proposed for molecular breeding and genotype-independent transformation of cotton, which implemented in concert, will avoid linkage drag and dependency on one or a few related lines with agronomic properties not up to current industry standards. The first strategy recommended is a switch to Max-R germplasm for development of transgenic cotton for input/output traits, now and in the foreseeable future (Wilkins and Mishra, supra). Strategies two and three entail introgression of RG alleles into the gene pool by using Max-R lines in breeding programs, coupled to positive selection for RG in advanced breeding lines.

What is claimed is:

1. A method for regenerating cotton, the method comprising:
(i) providing a cotton explant selected from the group consisting of Gossypium,
(ii) inducing callus formation in an induction medium comprising two or more auxins,
(iii) selecting superior callus, and
(iv) culturing the superior callus to form embryogenic callus.

2. The method of claim 1, wherein the explants are selected from the group consisting of hypocotyl, cotyledon, immature zygotic embryos, leaves, anthers, petal, ovules, roots, meristems and mixtures thereof.

3. The method of claim 1, wherein the cotton is an Acala cotton variety.

4. The method of claim 3, wherein the Acala cotton variety is selected from the group consisting of Maxxa, Riata, and Ultima.

5. The method of claim 1, wherein the two auxins are selected from the group consisting of dichlorophenoxyacetic acid (“2,4-D”) and α-napthaleneacetic acid (“NAA”).

6. The method of claim 5, wherein 2,4-D is present in the medium in concentrations between about 0.025 mg/L and about 0.1 mg/L.

7. The method of claim 6, wherein 2,4-D is present in the medium at about 0.05 mg/L.

8. The method of claim 7, wherein 2,4-D is present in the medium at about 0.1 mg/L.

9. The method of claim 5, wherein NAA is present in the medium in concentrations between about 1.5 mg/L and about 5 mg/L.

10. The method of claim 9, wherein NAA is present in the medium at about 1.5 mg/L.

11. The method of claim 9, wherein NAA is present in the medium at about 2 mg/L.

12. The method of claim 1, wherein the medium is free of cytokinins.

13. The method of claim 1, wherein the medium is Murashige and Skoog medium and the carbohydrate source is glucose or sucrose.

14. The method of claim 13, wherein the carbohydrate source is glucose and the glucose is at 30 g/L.

15. The method of claim 1, further comprising transferring the embryogenic callus to a plant germination medium and culturing the embryogenic callus on the plant germination medium until a plantlet is formed.

16. The method of claim 15, further comprising rooting the plantlet and developing fertile plants and seeds.

17. The method of claim 15, wherein the plant germination medium is Stewart's medium.

18. The method of claim 1, wherein the callus is induced in light-dark cycles of about 16 hours of light and about 8 hours of darkness at a temperature from about 25 degrees Celsius to about 35 degrees Celsius.

19. The method of claim 18, wherein the temperature is from about 26 degrees Celsius to about 30 degrees Celsius.

20. The method of claim 1, wherein the callus is induced in induction medium for about four to about six weeks.

21. The method of claim 1, wherein the step of culturing the superior callus to form embryogenic callus includes filtering and washing the cultures every two to three weeks.

22. A method for transforming cotton, the method comprising:
(i) providing a cotton explant selected from the group consisting of Gossypium,
(ii) inducing callus formation in induction medium,
(iii) suspending callus in suspension culture to break up the callus,
(iv) injuring cells to produce single cells and small cell clusters,
(v) co-cultivating the cells with Agrobacterium,
(vi) culturing cells under selection to select against Agrobacterium, and
(v) recovering transgenic cells.

23. The method of claim 22, wherein the explants are selected from the group consisting of hypocotyl, cotyledon, immature zygotic embryos, leaves, anthers, petals, ovules, roots, meristems and mixtures thereof.

24. The method of claim 22, wherein the cotton is an Acala cotton variety.

25. The method of claim 22, the method further comprising regenerating a cotton plant.

26. A cotton plant produced by a method comprising
(i) providing a cotton explant derived from an elite cotton species selected from the group consisting of Gossypium hirsutum L.,
(ii) inducing callus formation in a medium comprising dichlorophenoxyacetic acid (“2,4-D”) and α-napthaleneacetic acid (“NAA”),
(iii) selecting superior callus, and
(iv) culturing the superior callus to form embryogenic callus.

27. A cotton plant produced by a method comprising
(i) providing a cotton explant selected from the group consisting of Gossypium,
(ii) inducing callus formation in induction medium,
(iii) suspending callus in suspension culture to break up the callus,
(iv) injuring cells to produce single cells and small cell clusters,
(v) co-cultivating the cells with Agrobacterium,
(vi) culturing cells under selection to select against Agrobacterium, and
(v) recovering transgenic cells.
